(19) United States
(12) Patent Application Publication
(10) Pub. No.: US 2021/0129143 A1
(43) Pub. Date: May 6, 2021

(54) SINGLE COPY LEVEL DETECTION OF ENTERIC VIRUSES

(71) Applicant: Arizona Board of Regents on Behalf of the University of Arizona, Tucson, AZ (US)

(72) Inventors: Jeong-Yeol Yoon, Tucson, AZ (US); Soo Chung, Tucson, AZ (US); Kelly A. Reynolds, Tucson, AZ (US)

(21) Appl. No.: 17/082,998

(22) Filed: Oct. 28, 2020

Related U.S. Application Data
(60) Provisional application No. 62/927,055, filed on Oct. 28, 2019.

(51) Int. Cl. B01L 3/00; C12Q 1/70

(52) U.S. Cl. CPC B01L 3/502715; B01L 2300/126; C12Q 1/701

(57) ABSTRACT

The invention provides methods, devices and kits for enteric virus detection using microfluidic paper analytic device (μPAD) without using any sample concentration or nucleic acid amplification steps, by directly imaging and counting on-paper aggregation of antibody-conjugated, fluorescent submicron particles.

### CROSS REFERENCE TO RELATED APPLICATIONS

This application is a U.S. nonprovisional application of International Application No. 62/927,055, filed Oct. 28, 2019, the contents of which are hereby incorporated by reference in their entirety.

### STATEMENT OF FEDERALLY SPONSORED RESEARCH AND DEVELOPMENT

This invention was made with government support under Grant No. 1361815, awarded by NSF. The government has certain rights in the invention.

### FIELD OF THE INVENTION

The field of the invention relates generally the measurement and/or detection of enteric viruses.

### BRIEF DESCRIPTION OF THE FIGURES

FIG. 1. Schematic illustration of norovirus assay (an exemplary embodiment) on μPAD using a smartphone-based fluorescence microscope. (a) 5 μL of norovirus solutions are added directly to the main channel of μPAD (made out of nitrocellulose), followed by 2 μL of anti-norovirus particle suspension (0.001% w/v). Solutions spread throughout the entire channel by capillary action, which are imaged by a smartphone-based fluorescence microscope. (b) A blue LED (480 nm) is irradiating the μPAD from the side. A smartphone with a microscope attachment and a bandpass filter (525±20 nm; green emission) captures the fluorescent images of a μPAD. Photograph courtesy of Soo Chung and Sean Perea. Copyright 2019.

FIG. 2. Benchtop microscope assay results for norovirus capsids. For each assay, 4 different areas of a single channel were imaged and analyzed to obtain the pixel counts of aggregated particles. The pixel counts from 4 different images were added together to yield a single data point. Only green channel images were used. Experiments were repeated 3 times (0-1 fg/μL) or 4 times (10 fg/μL−10 pg/μL), each time using a different μPAD. Error bars represent standard errors of such 3 to 4 assays. * indicates statistically significant difference (p<0.05 with Wilcoxon rank sum test) from a negative control sample. Left: representative raw, background-removed, and non-aggregated particles-removed images (captured by a benchtop fluorescence microscope and processed with ImageJ) of a μPAD at given norovirus capsid concentrations. These images are zoomed-in versions (400 μm×400 μm) to clearly show the particles; the actual images used in the assays are 1.060 mm wide and 0.792 mm long. Right: average pixel counts from μPAD are plotted against norovirus capsid concentrations, using a benchtop fluorescence microscope and ImageJ processing.

FIG. 3. Specificity test. Three different concentrations of Zika virus and norovirus were tested with anti-norovirus conjugated particles. Benchtop microscope assays and ImageJ analyses were used. Other experimental conditions are identical to those shown in FIG. 2.

FIG. 4. Smartphone assay results for intact norovirus in DI water. For each assay, 4 different areas of a single channel were imaged and analyzed to obtain the pixel counts of aggregated particles. The pixel counts from 4 different images were added together to yield a single data point. Both green and red channels were combined to maximize pixel intensities. Experiments were repeated 3 times, each time using a different gAD. Error bars represent standard errors of such 3 assays. Wilcoxon rank sum test was performed and * indicates statistically significant difference (p<0.05) from a negative control sample. Left: representative raw and processed images of gAD at given intact norovirus concentrations. These images are zoomed-in versions (196 μm×196 μm) to clearly show the particles. Right: Average pixel counts from gAD are plotted against intact norovirus concentrations, using a smartphone-based fluorescent microscope and a MATLAB code.

FIG. 5. Smartphone assay results for tap water. Other experimental conditions are identical to those shown in FIG. 3, except that the assays were repeated 6 times.

FIG. 6. Smartphone assay results for reclaimed wastewater. Other experimental conditions are identical to those shown in FIG. 3, except that the assays were repeated 6 times.

FIG. 7. Image processing algorithm using ImageJ for benchtop fluorescence microscopic images (left) and MATLAB GUI code for smartphone fluorescence microscopic images (right). Using the pre-determined cut-off pixel intensity (to remove background) and pixel area (to isolate aggregated particles), along with binarization, a processed image is generated showing only the aggregated particles. The total pixel counts are added altogether from 4 different images from a single gAD channel, which makes up a single data point. This experiment is repeated 3-6 times, each time using a different gAD, to evaluate the average pixel counts. Images in the first and last columns are raw images; those in the second and third columns are zoomed-in versions to clearly show the particles.

FIG. 8. Concentration of norovirus directly on the μPAD. 10 mL of norovirus solutions are concentrated directly on μPAD (nitrocellulose) by encasing the μPAD into a syringe filter holder.

FIG. 9. Experiments are repeated for norovirus capsids (again using a benchtop fluorescent microscope and ImageJ processing), with a concentration method.

FIG. 10. The smartphone based fluorescence microscope is further improved using the 3D-printed enclosure, where a microscope attachment to a smartphone, a blue LED light source, a button battery, and a chip holder are incorporated.

FIG. 11. Representative raw (left), background-removed (middle), and non-aggregated particles-removed images (right) of a μPAD. Images were captured by a benchtop fluorescence microscope (top) and a benchtop light microscope, both processed with ImageJ.

FIG. 12. The user interface of an original, MATLAB GUI app. A raw smartphone image is loaded, and a square crop is applied circumscribing the circular microscopic field of view. Using the pre-determined cut-off pixel intensity and pixel area, along with binarization, a processed image is generated showing only the aggregated particles.

FIG. 13. Smartphone assay results for 0.5 ppm (left) and 5 ppm (right) chlorine added to DI water. Other experimental conditions are identical to those shown in FIG. 3.

### BACKGROUND

Enteric viruses are small infectious agents that can cause gastrointestinal disease upon ingestion of very low doses. These viruses may be present naturally in aquatic environments or, more commonly, are introduced through human activities such as leaking sewage and septic systems, urban runoff, agricultural runoff, and, in the case of estuarine and marine waters, sewage outfall and vessel wastewater discharge. Over 100 types of pathogenic viruses are excreted in human and animal wastes. These viruses can be transported in the environment through groundwater, estuarine water, seawater, rivers, aerosols emitted from sewage treatment plants, insufficiently treated water, drinking water, and private wells that receive treated or untreated wastewater either directly or indirectly. These viruses, collectively known as enteric viruses, usually are transmitted via the fecal-oral route and primarily infect and replicate in the gastrointestinal tract of the host. Enteric viruses are shed in extremely high numbers in the feces of infected individuals, typically between 10⁵ and 10¹¹ virus particles per gram of stool.

Detection of enteric viruses requires an extremely low limit of detection (LOD), especially when assessing viruses in reclaimed wastewater or unconfined aquifers used as sources of drinking water. Norovirus is one of such well-known examples and is the most common cause of epidemic and sporadic gastroenteritis worldwide.

Like norovirus, many other viruses use the enteric tract as a route of entry to the human, animal or avian host. The onset of acute enteritis is associated with infection by viruses that replicate at or near the site of entry into the intestinal mucosa, including caliciviruses, rotaviruses, adenoviruses, astroviruses, and coronaviruses. These ‘enteric’ viruses occur globally and share similar features. See e.g., Bishop R F, Kirkwood C D. Enteric Viruses. Reference Module in Biomedical Sciences. 2014; B978-0-12-801238-3.02566-6. For example, SARS-CoV-2 virus has been detected in feces. See Ding and Liang, Gastroenterology 159:53-61 (2020). Furthermore, SARS-CoV-2 RNA was detected in the endoscopic specimens of the esophagus, stomach, duodenum, and rectum from several patients. Lin L, Jiang X, Zhang Z, et al. Gastrointestinal symptoms of 95 cases with SARS-CoV-2 infection. Gut 2020; 69:997-1001. Moreover, the first reported case of COVID-19 in the United States experienced diarrhea and tested positive for viral RNA in his feces but not serum. See, Holshue M L, DeBolt C, Lindquist S, et al. First case of 2019 novel coronavirus in the United States. N Engl J. Med 382:929-936 (2020).

Studies have indicated that norovirus infection can occur upon exposure to as few as 18 virions. Highly sensitive detection methods are needed for assessing exposure to norovirus, especially considering that the methods for virus recovery and concentration from environmental matrices are rather inefficient. In addition, the infectivity of human noroviruses by in vitro cell culture has proven to be quite complex (only possible in stem cell-derived human enteroids), which prevents the use of traditional culture-based assays for evaluating virus infectivity in environmental matrices. Due to this limitation, norovirus has been assayed by either reverse transcription polymerase chain reaction (RT-PCR) or sandwich immunoassay techniques.

While RT-PCR-based techniques do provide necessary specificity for detection and identification of enteric viruses (such as norovirus), these molecular methods are susceptible to inhibition by multiple components associated with environmental matrices and fail to provide sufficient rapidity and field-applicability. Immunoassay techniques are simpler than RT-PCR and have the potential to be incorporated on a microfluidic platform. Specifically, microfluidic paper analytic devices (μPADs) have shown numerous advantages over silicone-based microfluidic devices, as they are lightweight, easy-to-fabricate via wax printing (no lithography), use spontaneous flow by capillary action, and have potential on-chip filtration capability. However, optical detection of low concentrations of pathogens has rarely been demonstrated on paper substrates, since paper is optically opaque and non-homogeneous (porous), generating substantial background scatter and reflection.

So far, single virus copy level detection of enteric viruses (such as norovirus) has rarely been demonstrated on paper substrates (including lateral flow assays and μPADs). While single copy level detection of other virus targets has indeed been demonstrated on paper substrates (20 copies of Ebola, 20 copies/μL of pseudorabies, and 1 copy/μL of HIV), all of them required nucleic acid amplifications, most notably isothermal methods such as loop-mediated isothermal amplification (LAMP). Such methods are not sufficiently simple for field-based applications (requiring a heater and thermostat system plus an expensive isothermal amplification kit) and cannot be considered near-real-time (just the amplification part can take from 15 minutes to 2.5 hours). As described previously, immunoassay on μPAD without sample concentration and/or nucleic acid amplification is the ideal method for field-based norovirus detection, which has unfortunately not been demonstrated at the single virus copy level. The LODs of paper-based norovirus immunoassays ranged from 10⁴ to 10⁶ copies/μL (=10 fg/μL to 1 pg/μL, as the weight of a single norovirus particle is approximately 10 ag considering its diameter of 35-40 nm) without concentration or amplification and 10² copies/μL with 1 hour reaction of signal amplification. The invention provides several benefits, for example, easy, inexpensive, yet extremely sensitive detection of an enteric virus such as norovirus and incorporates use smartphone which makes testing portable and can be useful in remote areas.

### PRIOR ART

Prior art documents are available which are concerned with measurement and/or various analytes to e.g. detect pathogens using various devices. For example, U.S. Pat. Nos. 10,613,082 B2, 10,132,802 B2, 8,889,424 B2, 10,054,584 B2 and 10,498,936 B2 relate to assay cassettes and testing devices that can be used to provide testing at the point of care. Furthermore, U.S. Pat. No. 10,352,920 B2 involves methods for monitoring and adjusting a physiological state of a subject are known in the art. U.S. Pat. No. 9,322,767 B2 relates to devices and methods for performing a point of care blood, cell, and/or pathogen count or a similar blood test). However, this method involves the use of a hemocytometer (a microscope glass slide with checkboard patterns to count the number of cells) instead of an LFIA cassette, they attempted to count the blood cell and pathogen (bacteria, but definitely not viruses). They did not use paper microfluidic device, nor a microscope

While these devices are suitable for testing of various analytes, they do not involve the use of a paper microfluidic device. This drawback limits their application makes them unsuitable for the detection of low levels of enteric viruses such as noroviruses or coronavirus.

### DESCRIPTION

One aspect of the invention pertains to a device for detecting and/or quantifying an enteric virus comprising a microfluidic paper analytic device (μPAD). In some embodiments of the invention, said virus tested is human enteric virus or virus is an animal (dog, cat, livestock, etc.) enteric virus. The virus tested may be norovirus or coronavirus.

In some embodiments, the device may further comprise a benchtop fluorescence microscope.

In other embodiments, wherein said device further comprises a smartphone-based fluorescence microscope comprising a smartphone, a microscope attachment, a light source (such as LED), a battery to power said light source (such as LED) (e.g., a button battery), and an optical filter.

Without being limited by theory, in some embodiments, the device may involve the use of a cover slip, which is added to “flatten” the paper chip as nitrocellulose paper tends to become curled occasionally.

The paper microfluidic chip may also be simply placed on a glass slide and placed into the device for smartphone fluorescence imaging. Optionally, cover slip is added to “flatten” the paper chip as nitrocellulose paper tends to become curled occasionally.

In further embodiments, the device may include a microscope attachment, an LED, a battery to power LED (e.g., a button battery), and an optical filter are housed within an enclosure to block ambient lighting. The enclosure may comprise plastic and/or metal (e.g., a plastic enclosure).

In some embodiments, multiple images may be taken from a single channel of a paper microfluidic chip, the exact positions of these images can be randomly chosen. For instance, four images may taken from a single channel of a paper microfluidic chip. Exact positions of these four images can be randomly chosen. In the further embodiments of the device, the slide that accommodates the paper chip has multiple “stops” to position the paper chip at multiple different fixed locations. This will make the user to position the chip in an easier and reproducible manner.

The microscope attachment of the device may comprise a bandpass filter or acrylic films (also known as “filter cards”).

Another aspect of the invention pertains to a method for detecting an enteric virus comprising
(a) applying a suspension comprising said virus to a microfluidic paper analytic device;
(b) adding an anti-virus conjugated fluorescent particle suspension to the microfluidic paper analytic device;
(c) allowing particles and viruses spread spontaneously throughout the μPAD channel via capillary action, allowing the particles to aggregate and facilitating imaging of individual particles.

In some embodiments, wherein the virus is present in a concentration ranging from 10⁰ to 10⁵ virions. The may be virus capable of causing disease upon ingestion of low doses ranging from 10⁰ to 10² virions.

Further, the method may include taking measurements without using any sample concentration or nucleic acid amplification step.

In some embodiments, the μPAD used in said method comprises nitrocellulose paper, cellulose paper, or polymeric fiber filter.

The method of embodiment 9, wherein said suspension has not been pre-purified, pre-concentrated, or pre-amplified prior to testing.

A further aspect of the invention encompasses a kit for detecting an enteric virus comprising
a microfluidic paper analytic device (μPAD),
a suspension of antibody conjugated fluorescent particles
optionally a syringe filter to concentrate a water sample, and
a smartphone-based fluorescence microscope.

In some embodiments, the invention pertains to a kit for detecting an enteric virus comprising

a device of the invention; and

one or more reagents (e.g., fluorescent particles conjugated with different antibodies) for carrying out detection and/or quantification of enteric viruses.

The following list of embodiments are mentioned by way of example:
1. A device for detecting and/or quantifying an enteric virus comprising a microfluidic paper analytic device (μPAD).
2. The device of embodiment 1, wherein said virus is a human enteric virus.
3. The device of embodiment 1, wherein said virus is an animal (dog, cat, livestock, etc.) enteric virus.
4. The device of embodiment 1, wherein said animal is dog, cat, livestock, etc.
5. The device of embodiment 1, wherein said virus is norovirus.
6. The device of embodiment 1, wherein said device further comprises a benchtop fluorescence microscope.
7. The device of embodiment 1, wherein said device further comprises a smartphone-based fluorescence microscope comprising a smartphone, a microscope attachment, a light source (such as LED), a battery to power said light source (such as LED) (e.g., a button battery), and an optical filter.
8. The device of embodiment 7, where a microscope attachment, an LED, a battery to power LED (e.g., a button battery), and an optical filter are housed within an enclosure to block ambient lighting. The enclosure may comprise plastic and/or metal (e.g., a plastic enclosure).
9. A method for detecting an enteric virus comprising
   (a) applying a suspension comprising said virus to a microfluidic paper analytic device;
   (b) adding an anti-virus conjugated fluorescent particle suspension to the microfluidic paper analytic device;
   (c) allowing particles and viruses spread spontaneously throughout the μPAD channel via capillary action, allowing the particles to aggregate and facilitating imaging of individual particles.
10. The method of embodiment 9, wherein the virus is present in a concentration ranging from 10⁰ to 10⁵ virions.
11. The method of embodiment 9, wherein said virus is capable of causing disease upon ingestion of low doses ranging from 10⁰ to 10² virions.
12. The method of embodiment 9, wherein said measurement is taken without using any sample concentration or nucleic acid amplification step.
13. The method of embodiment 9, wherein said μPAD comprises nitrocellulose paper, cellulose paper, or polymeric fiber filter.
14. The method of embodiment 9, wherein said suspension has not been pre-purified, pre-concentrated, or pre-amplified prior to testing.
15. The method of embodiment 9, wherein said method involves a single virus copy level detection of said virus.
16. The method of embodiment 9, wherein said virus is a human enteric virus.
17. The method of embodiment 9, wherein said virus is an animal (dog, cat, livestock, etc.) enteric virus.
18. The method of embodiment 9, wherein said animal is dog, cat, livestock, etc.
19. The method of embodiment 9, wherein said virus is norovirus.
20. The method of embodiment 9, wherein said imaging and counting aggregation of antibody-conjugated, fluorescent submicron particles is on-paper.
21. The device of embodiment 1, wherein said method involves a single virus copy level detection of said virus.
22. The method of embodiment 1, wherein said sample comprises water.
23. A kit for detecting an enteric virus comprising a microfluidic paper analytic device (μPAD), a suspension of antibody conjugated fluorescent particles optionally a syringe filter to concentrate a water sample, and a smartphone-based fluorescence microscope.
24. A kit for detecting an enteric virus comprising
   (a) a device of claim 1; and
   (b) one or more reagents (e.g., fluorescent particles conjugated with different antibodies) for carrying out detection and/or quantification of enteric viruses.
25. The kit of claim 23, wherein said virus is norovirus.
26. The method of embodiment 9, wherein said antibody is a polyclonal antibody.
27. The method of embodiment 9, wherein the fluorescent particle is a fluorescent polystyrene particle.
28. The method of embodiment 9, wherein further comprises:
   (1) fabricating a microfluidic paper analytic device (e.g., μPAD) with multiple channels on it for simultaneously conducting multiple assays;
   (2) conjugating an antibody to fluorescent particles to obtain an anti-virus conjugated fluorescent submicron particle suspension;
   wherein said steps are performed prior to said steps (a)-(c).
29. The method of embodiment 9, wherein further comprises:
   (i) imaging the aggregation of anti-virus conjugated fluorescent particles;
   (ii) removing the background noises and autofluorescence from paper substrate using an optimized threshold intensity and isolating only the fluorescent particles;
   (iii) binarizing an entire image;
   (iv) removing the smaller size of particles to isolate only the aggregated particles;
   (v) relating the total pixel area to the virus concentration to construct a standard curve and estimate the virus concentration from an unknown sample;
   wherein said steps are performed after said steps (a)-(c).
30. A method for detecting an enteric virus comprising
   (a) fabricating a microfluidic paper analytic device (e.g., μPAD) with multiple channels on it for simultaneously conducting multiple assays;
   (b) conjugating an antibody to fluorescent particles to obtain an anti-virus conjugated fluorescent submicron particle suspension;
   (c) applying a suspension comprising said virus to the microfluidic paper analytic device;
   (d) adding said anti-virus conjugated fluorescent particle suspension to the microfluidic paper analytic device;
   (e) allowing particles and viruses spread spontaneously throughout the μPAD channel via capillary action, allowing the particles to aggregate and facilitating imaging of individual particles;
   (f) imaging the aggregation of anti-virus conjugated fluorescent particles;
   (g) removing the background noises and autofluorescence from paper substrate using an optimized threshold intensity and isolating only the fluorescent particles;
   (h) binarizing an entire image;
   (i) removing the smaller size of particles to isolate only the aggregated particles;
   (j) relating the total pixel area to the virus concentration to construct a standard curve and estimate the virus concentration from an unknown sample.
31. A kit for detecting an enteric virus comprising a microfluidic paper analytic device (μPAD), antibody conjugated fluorescent particles optionally a syringe filter to concentrate a water sample, and a smartphone-based fluorescence microscope.
32. A kit for detecting an enteric virus comprising a microfluidic paper analytic device (μPAD), antibody conjugated fluorescent particles.
33. The device of embodiment 7, wherein said optical filter is a bandpass filter or acrylic films (also known as “filter cards”).

### Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

As used herein, the term “human enteric virus” includes viruses that belong to the families Picornaviridae (e.g., polioviruses, enteroviruses, coxsakieviruses, hepatitis A virus, and echoviruses), Adenoviridae (e.g., adenoviruses), Caliciviridae (e.g., noroviruses, caliciviruses, astroviruses, and small round-structured viruses), and Reoviridae (e.g., reoviruses and rotaviruses).

As used herein, the term “antibody” refers to an antibody to the enteric virus being detected. For example, Rabbit polyclonal antibody to norovirus capsid protein VP1 (anti-norovirus) may be used as an antibody in the detection of norovirus.

As used herein, the term “fluorescent particle” refers to a polymeric particle (e.g., a nanoparticle or microparticle) attached a fluorescent dye, such as a fluorescent polystyrene particle, more preferably carboxylated, yellow-green fluorescent, polystyrene particles. For example, fluorescent particle may have a diameter in the range of about to 0.2 μm to about 2 μm (or about to 0.2 μm to about 1 μm; or about to 1 μm to about 2 μm; or about to 0.4 μm to about 0.8 μm; or about to 0.4 μm to about 0.6 μm; or 0.2 μm to about 0.7 μm). In some embodiments, the diameter may be 0.5 μm.

### EXAMPLES

Methods

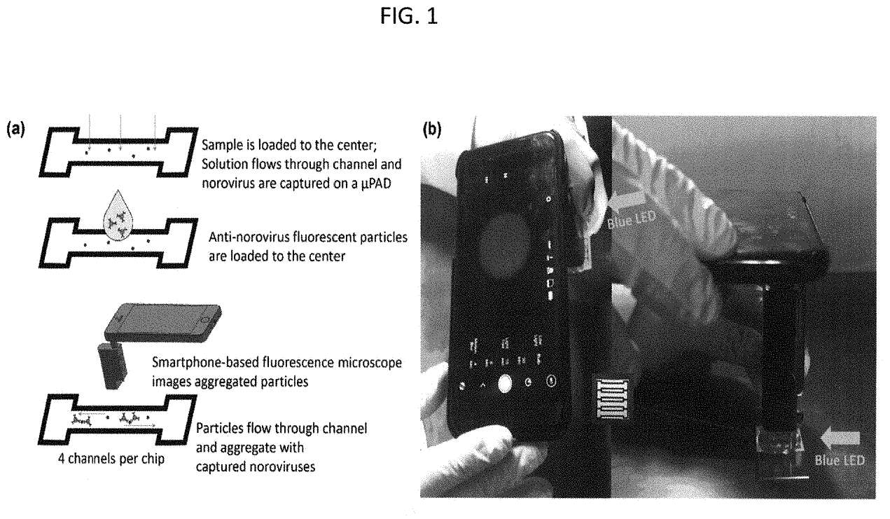

μPAD Fabrication. A ColorQube wax printer (Xerox Corporation; Norwalk, Conn., USA) was used to print the microfluidic design (FIG. 1a) onto a nitrocellulose paper (Hi-Flow™ Plus Membrane, catalog number HF07502XSS; Millipore; Billerica, Mass., USA). Each chip has four wax-printed channels (21 mm long and 2.4 mm wide). Each chip was heated on a hot plate (Corning; Corning, N.Y., USA) at 120° C. until the surface-printed wax was melted to fill the paper pores underneath.

Antibody Conjugation to Fluorescent Particles. Rabbit polyclonal antibody to norovirus capsid protein VP1 (anti-norovirus, catalog number ab92976; Abcam, Inc.; Cambridge, Mass., USA) was used for assaying both norovirus capsids and intact noroviruses. Anti-norovirus was covalently conjugated to carboxylated, yellow-green fluorescent, polystyrene particles (particle diameter=0.5 μm; Magsphere, Inc.; Pasadena, Calif., USA). The fluorescent characteristics of these particles were reported by the manufacturer: maximum excitation at 480 nm (blue) and maximum emission at 525 nm (green). Prior to antibody conjugation, particles were pre-washed with deionized (DI) water to remove surfactants from the stock solution, through centrifuging at 9.9 G for 13 minutes. The antibody was then conjugated to these fluorescent particles using a method known in the art.

Norovirus Sample Preparation. Initially, recombinant norovirus group-1 capsid (MyBiosource, Inc.; San Diego, Calif., USA) was used as a target. Norovirus capsids were serially diluted in DI water from the 1 ng/μL stock solution to make 10 pg/μL, 1 pg/μL, 100 fg/μL, 10 fg/μL, 1 fg/μL, 100 ag/μL, 10 ag/μL, and 1 ag/μL, all in 1 mL volume at 1:10 dilution each (4-10 serial dilutions). The systematic errors of pipettes were ±0.8% for a 1,000 μL pipette and ±0.6% for a 100 μL pipette, resulting in the propagated errors of 2.0%-3.1% for the given range of dilutions. These errors were too small to be represented as the horizontal error bars in the logarithmic scale x-axes in all plots.

Intact norovirus samples were collected from toilet fecal samples during an active norovirus outbreak. These samples were confirmed and quantified by quantitative reverse transcription polymerase chain reaction (RT-qPCR). Fecal samples were suspended in sterile phosphate buffered saline (PBS) solution (pH 7.4) at 10% w/v. These fecal suspensions were centrifuged at 1,455 G for 10 minutes using Centriprep centrifugal filters (50 kDa cutoff; EMD Millipore, Burlington, Mass., USA) to purify virus particles. The retentates (˜0.75 mL) were divided into aliquots of 200 μL and frozen or subjected to nucleic acid extraction. To confirm and quantify norovirus, virus nucleic acids were extracted using the QIAmp viral RNA extraction kit (Qiagen, Chatsworth, Calif., USA) and RT-qPCR assays were performed for three different genogroups of norovirus (GI, GII, and GIV) following previously reported assays. Gil norovirus RNA was predominantly detected from the fecal suspensions, with a viral load of approximately 10⁷ virus targets per mL of stool supernatant. These fecal suspensions were serially diluted in various water samples (described in the following section) from the 10000 genome copies/μL to obtain 1000 genome copies/μL, 100 copies/μL, 10 copies/μL, and 1 copy/μL, again all in 1 mL volume at 1:10 dilution each (1-4 serial dilutions). Using the same systematic errors of pipettes, the propagated errors were 1.0%-2.0% for the given range of dilutions. Again, these errors were too small to be represented as the horizontal error bars in the logarithmic scale x-axes in all plots.

Specificity Test. Zika virus (attenuated virus particles; Natural Zika Virus Range Verification Panel; Zepto Metrix Corporation, Buffalo, N.Y., USA) was used to evaluate the cross-reactivity of anti-norovirus with this assay. Both norovirus and Zika virus are single-stranded RNA viruses, have globular shapes, and are similar in size. Identical experiments were performed by substituting norovirus samples with Zika virus samples. The concentrations of Zika virus samples were 1.6 pg/μL, 200 fg/μL, and 20 fg/μL.

Water Samples. Various types of environmental water samples, spiked with known concentrations of norovirus, were tested in this work: deionized (DI) water, drinking tap water, and reclaimed wastewater. The latter was produced in a facility utilizing primary sedimentation dissolved air flotation, four parallel five-stage Bardenpho processes, disk filtration, and chlorination. These water samples were tested for pH, conductivity, and chlorine residual. pH was measured using the pH electrode and pH monitor (Pinpoint American Marine Inc.; Ridgefield, Conn., USA). Conductivity was measured using the UltraPen PT1 (Myron L Company; Carlsbad, Calif., USA). Free chlorine residual was assayed by the EPA-accepted Thermo Orion Method AC4P72 (using N, N-diethyl-p-phenylenediamine, thus known as DPD method; Thermo Fisher, Waltham, Mass., USA) by measuring absorbance at 520 nm using a miniature spectrophotometer (USB4000, Ocean Optics, Inc.; Dunedin, Fla., USA).

Assay Procedure. Norovirus suspensions (5 μL) from spiked environmental water samples were pipetted directly to the center of each μPAD channel made out of nitrocellulose paper, without using any pre-treatments. This norovirus suspension spread through each microfluidic channel, where norovirus particles were captured onto nitrocellulose paper (polarity filter) via electrostatic interactions. After loading norovirus, 2 μL of anti-norovirus conjugated fluorescent polystyrene particle suspension (0.001% w/v for DI water and 0.002% w/v tap water and reclaimed wastewater) were loaded onto the center of each channel on the μPAD where noroviruses were captured (FIG. 1a). Anti-norovirus conjugated particles flowed through and filled the entire channel by capillary action (or wicking). These particles were aggregated by antibody-antigen binding, i.e., immunoagglutination, which were imaged as described in the following section.

Imaging Particle Aggregation on μPADs Using a Benchtop Fluorescence Microscope. Particle aggregation with norovirus was imaged by taking 4 random images of each channel with a 5-second exposure time, initially using a benchtop fluorescence microscope (Eclipse TS 100; Nikon Corp.; Tokyo, Japan), equipped with a fluorescence filter (AG Heinze B-2E/C; A.G. Heinze, Inc.; Lake Forest, Calif., USA) and an imaging software (NIS Elements; Nikon Corp.; Tokyo, Japan). Only green channel images were used. From the processed images, the pixel counts were evaluated, which were added together for 4 different images to yield a single data point. This procedure was repeated 3-4 times, each time using a different μPAD.

Imaging Particle Aggregation on μPADs Using a Smartphone-based Fluorescence Microscope. The smartphone-based fluorescence microscope (FIG. 1b) consisted of an external microscope (XFox Professional 300× Optical Glass Lenses; X&Y Ind., Shenzhen, China) with magnification 200×-300×, attached to a smartphone (iPhone 7; Apple, Inc.; Cupertino, Calif., USA). A blue excitation light source was provided by a secondary smartphone flashlight with a 480±10 nm bandpass filter (catalog number 43-115; Edmund Optics, Barrington, N.J., USA). This can be easily replaced by any blue LED. An unmounted 525±20 nm bandpass filter (catalog number BP525-D25; Midwest Optical Systems, Inc.; Palatine, Ill., USA) was placed in between the μPAD and the objective lens of a microscope to capture green fluorescence emission. All images were taken using the ProCam4 app (Samer Azzam, http://www.procamapp.com; downloaded via iTunes), where the exposure time and white balance could be manually adjusted. Light trail exposure time was 4 seconds, white balance was 4000, and ISO was 200. Similar to benchtop fluorescence microscopy, 4 images were taken from each channel to yield a single data point. Experiments were repeated 3-6 times, each time using a different μPAD.

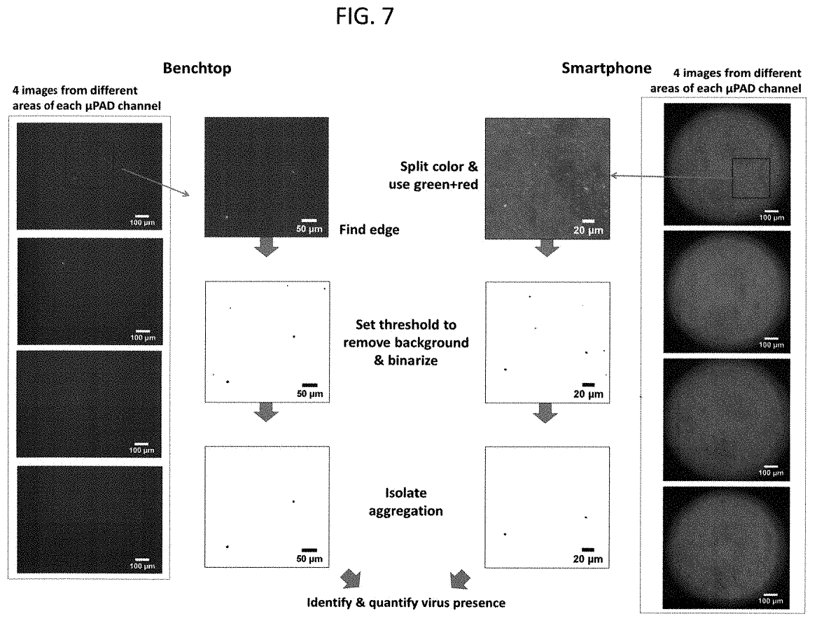

Image Analysis for Benchtop Fluorescence Microscopic Images. ImageJ (U.S. National Institutes of Health; Bethesda, Md., USA) was initially processed on a separate desktop computer to analyze the images taken on a benchtop fluorescence microscope. For benchtop fluorescence microscopic images, ‘Find Edges’ option in ImageJ was utilized to outline the image of particles. All pixels with intensity values <100 (out of 255 for green emission) were considered background noise and eliminated. This threshold value (100) was determined by comparing the images with those measured by a higher magnification fluorescence microscope. All other pixels with intensity values >100 were selected, the interior of the edges were filled, and these selected pixels were binarized. This procedure resulted in binary images of the particles. Once the images were binarized, ‘Analyze Particles’ function was selected in ImageJ, and the pixel area was obtained. The pixel area <50 was eliminated since they were single particles that were not aggregated by norovirus. This threshold value (50) was determined by comparing the images to those measured by a higher magnification fluorescence microscope. The final data consisted of the following: 1) the number of aggregated particle clusters, and 2) the total accumulated pixel counts of all aggregated particles, for the given image. This procedure is schematically illustrated in FIG. 7.

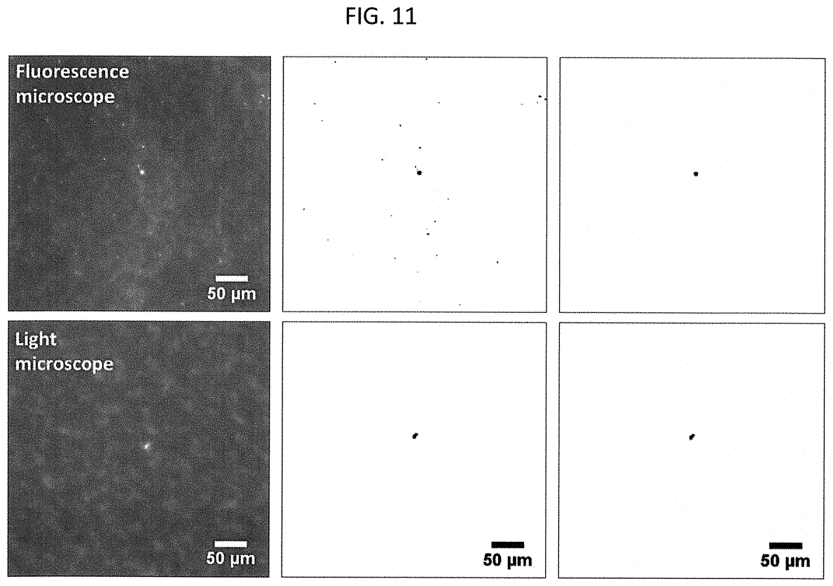

Image Analysis for Smartphone-based Fluorescence Microscopic Images. All smartphone-based fluorescence microscopic images were split into red, green, and blue channels. While the maximum emission wavelength of the fluorescent particles was 525 nm, their emission is actually ranged over 550 nm, i.e. boundary of green and red colors (hence they are referred as “yellow-green” particles). Therefore, their fluorescence emission could be captured in not only green but also red channels. Since nitrocellulose paper absorbed and scattered light at most wavelengths (its color is bright white) and the maximum exposure time of a smartphone camera was much shorter than that of a benchtop fluorescence microscope, the pixel intensities were quite low. Therefore, both green and red channels were combined to maximize the pixel intensities. Unlike the benchtop fluorescence microscopy, the mean pixel intensities of combined green and red channel images were evaluated using an original code developed in MATLAB version R2017a (The Mathworks, Inc.; Natick, Mass., USA). A graphical user interface (GUI) (FIG. 11) was created and used to automate the analysis procedure and to provide its user-friendliness.

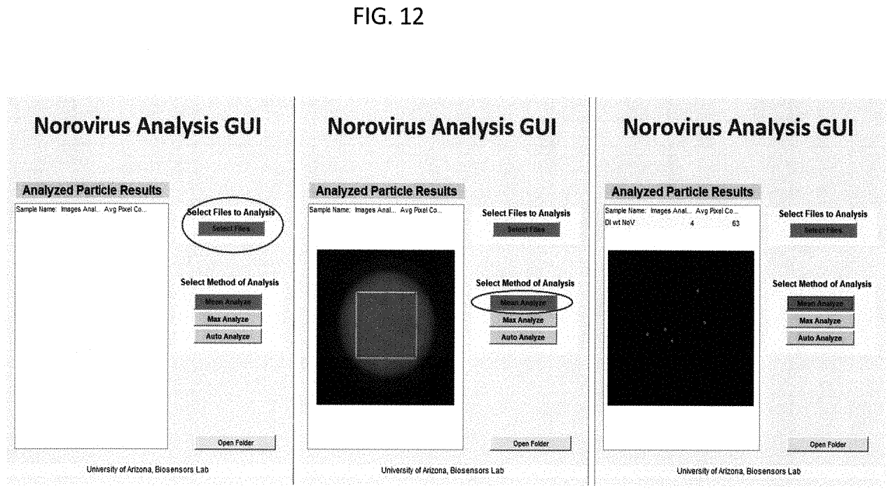

Smartphone microscopic images were processed using a similar algorithm to the benchtop fluorescence microscopy and ImageJ processing. Since the bright field views of smartphone microscopic images were circular in shape, all images were cropped into squares circumscribing those circles, such that all pixels could be utilized for analyses. Aggregated fluorescent particles always exhibited the combined green and red pixel intensities substantially higher than the overall mean intensities of the cropped area. To eliminate background noise and isolate only the particles, cut-off intensities were applied to the images set at overall mean intensity +40-50. The resulting images were then binarized. To eliminate the non-aggregated particles, those with a pixel area <30 were eliminated from the binarized images. This cut-off value of a 30 pixel area was smaller than that of benchtop fluorescence microscopy, 50, due to the lower magnification and narrower dynamic range of smartphone-acquired images. This threshold filtering successfully eliminated all ambient light variations, indicating that the method is appropriate for field use. Again, this procedure is schematically illustrated in FIG. 7. The MATLAB GUI generated the accumulated pixel counts of all aggregated particles, for the given image. The MATLAB code and its GUI were adapted to be executed within MATLAB Mobile (The Mathworks, Inc.; Natick, Mass., USA), to enable the image analysis performed within a smartphone (FIG. 12). Once images were acquired, the total assay time was less than one minute including the time for user input.

Statistical Analysis. 4 different images were taken from each μPAD channel (FIG. 1a) and the sum of pixel counts from these 4 images (representing the extent of particle aggregation) were recorded for the given concentration of norovirus. These experiments were repeated 3-6 times for each concentration of norovirus, each time using different gAD. Averages of these 3-6 gAD assays were recorded. P values for each norovirus concentration against the negative control sample (unspiked) were calculated using Wilcoxon rank sum test, performed with JMP software version 14.3.0 (SAS Institute, Inc.; Cary, N.C., USA) with α=0.05.

Results and Discussion

Benchtop Microscope Assays. Initially, μPAD assays were conducted for assessing the norovirus capsids, using a benchtop fluorescence microscope and subsequent ImageJ analysis. All serial dilutions were made in 1 mL volume and vortex-mixed to ensure that there were sufficient amounts of norovirus in each dilution even at the lowest concentration. For each assay, 4 different areas of a single channel were imaged. Through size analysis, the locations of fluorescent particles (both non-aggregated and aggregated) could easily be determined, which showed the pixel intensities of at least 100 (out of 255). Distinction could also be made between non-aggregated and aggregated particles using the pixel area of 50. Therefore, the raw images were processed to eliminate the pixels with <100 intensity (to remove background) and the pixel area <50 (to remove non-aggregated particles).

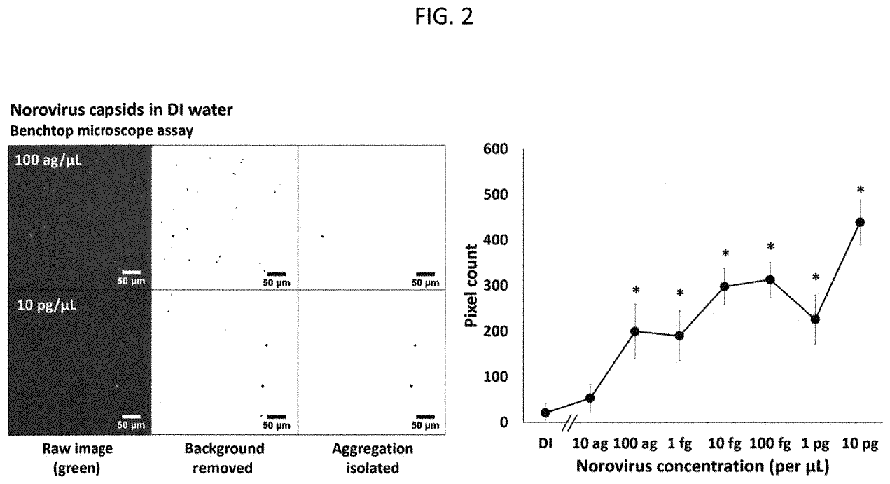

From these 4 processed images from a single μPAD channel, the number of pixels were added together to yield a single data point. This number corresponded to the extent of particle aggregation and thus norovirus concentration. Experiments were repeated 3-4 times, each time using a different gAD. Representative zoomed-in images (raw and processed) are provided in FIG. 2 to the left to better represent the aggregated particles. To confirm whether the pixel area truly represented the particle size and distinguished the aggregated from non-aggregated particles, fluorescence and light microscopic images were obtained for the aggregated particles on a μPAD and processed in the same manner (FIG. 11). Two different types of particles were observed in fluorescence images, where the smaller ones potentially represent the non-aggregated particles and the bigger ones the aggregated particles.

Note that the particle size (0.5 μm) is comparable to the emission wavelength (525 nm) of fluorescent particles. With light microscopic images, however, only the bigger particles could be observed, exactly at the same locations of bigger sized particles in the fluorescence microscopic images. As the particle size (0.5 μm) is smaller than the upper limit of visible wavelength (400-750 nm), it will be difficult to image the 0.5-μm, non-aggregated particles, while the aggregated particles (>0.8 μm) can be imaged relatively easily.

The averages and standard errors of these pixel counts from 3-4 independent assays were plotted against the norovirus concentration in FIG. 2 to the right. As sample size is relatively small, it was difficult to assume normal distribution for each data point. Therefore, a nonparametric Wilcoxon rank sum test was conducted for each data point in comparison to the zero-concentration data point (in DI water) as a negative control. The lowest concentration of norovirus capsid that passed the Wilcoxon rank sum test (p<0.05) was 100 ag/μL, which is the LOD of this assay.

All concentrations from 100 ag/μL to 10 pg/μL were also significantly different from the zero concentration (negative control), indicating the particle aggregation was highly correlated to the norovirus presence and minimum non-specific aggregation. This LOD is several orders of magnitude lower than 0.25-12.5 pg/μL (=ng/mL) with the commercial lateral flow assays (including immunoCatch-Noro from Eiken Chemical, GE test Noro Nissui from Nissui Pharmaceutical, and Quick Navi-Noro 2 from Denka Seiken) and 10-100 fg/μL as reported in the recent literature utilizing nanostructures as well as laboratory equipment such as a microplate reader or surface plasmon resonance equipment. Since the weight of a single norovirus particle is approximately 10 ag considering its diameter (35-40 nm), this LOD value is close to a single virus particle level within an order of magnitude.

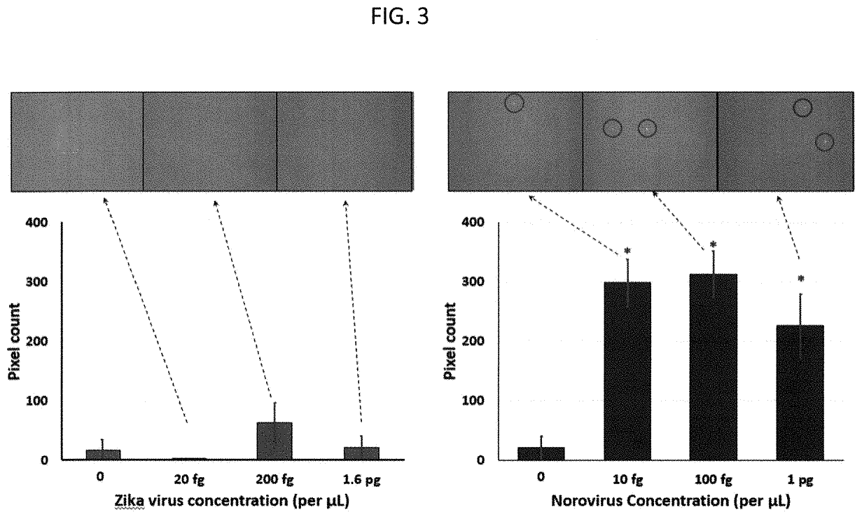

Specificity Test. To evaluate the specificity of this assay, Zika virus was assayed using anti-norovirus conjugated particles and compared with the results of norovirus assay. Experimental conditions were identical to those of norovirus assays. As shown in FIG. 3, the pixel counts were much smaller with Zika virus than with norovirus. All Zika virus concentrations were not significantly different from the zero concentration (negative control) using nonparametric Wilcoxon rank sum test. Taken together these results, satisfactory specificity was achieved by the assay at least for the given experimental conditions.

Smartphone Microscope Assays. Next, the same experiments were repeated while replacing norovirus capsids with intact noroviruses (refer to the Methods section for the preparation of intact norovirus and RT-qPCR assay). The μPAD assays were conducted for assessing intact norovirus using a smartphone microscope shown in FIG. 1b and MATLAB Mobile GUI app (FIG. 12). Intact noroviruses were initially diluted in deionized (DI) water. Again, all serial dilutions were made in 1 mL volume and vortex-mixed to ensure that there were sufficient amounts of norovirus even at the lowest concentration (1,000 genome copies in 1 copy/μL sample). Since the smartphone constantly attempts to compensate for lighting bias and exposure, and to adjust white balance, the overall brightness of raw images was different from assay to assay. Therefore, the raw images (already square-cropped circumscribing circular field of view) were processed to eliminate the pixels with the intensities smaller than the overall mean+50 (out of 255; to remove background noise), binarized, and further processed to eliminate the pixel areas smaller than 30 (to remove non-aggregated particles).

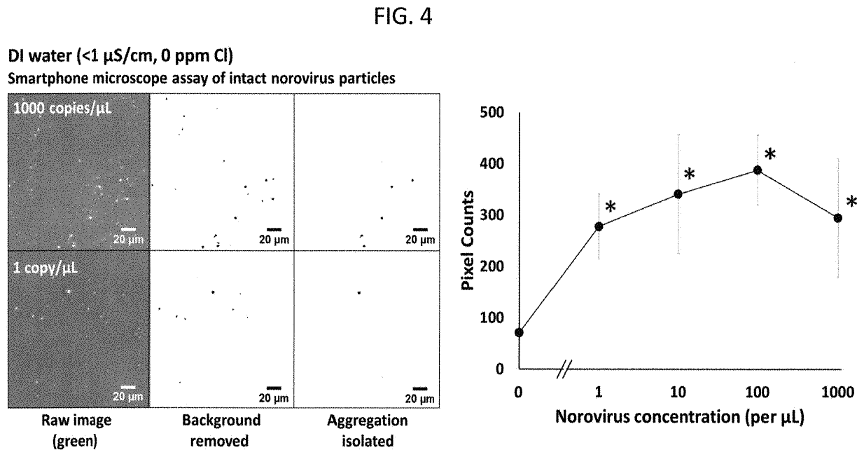

Refer to the Methods section for details. Similar to the benchtop microscope assays, 4 different areas of a single channel were imaged and analyzed, and the pixel counts were added together to yield a single data point. Experiments are repeated 3 times, each time using a different μPAD. The results are depicted in FIG. 4, showing the representative, zoomed-in images (raw, background removed, and aggregation isolated) for 1 copy/μL (the lowest concentration assayed) and 1000 copies/μL (the highest concentration significantly different from the negative control, i.e., virus-free deionized water) to the left, and the plot of average pixel counts against the norovirus concentration (genome copies per μL) to the right

The lowest concentration that is significantly different (p<0.05 with Wilcoxon rank sum test) from the control (virus-free DI water) is 1 copy/μL, the LOD of this assay. It corresponds to 10 ag/μL considering the size of a norovirus particle, 35-40 nm, and is one order of magnitude lower than that of assaying norovirus capsids, 100 ag/μL. This can be attributed to the fact that the norovirus capsids were recombinant proteins that might have inferior affinity to the anti-norovirus compared to the intact norovirus samples. Concentrations of 10 and 100 copies/μL are also significantly different from the control (p<0.05). The average pixel counts at the highest concentration, 1000 copies/μL, is slightly smaller than that of 100 copies/μL, indicating that this concentration is outside the linear range of assay. In other words, there were too many virus particles that “consumed” all antibodies, which subsequently failed to connect antibody-conjugated particles together. Despite this, it is still substantially higher than the negative control (p<0.05).

To further confirm this extremely low LOD of 1 copy/μL, the number of aggregated particle clusters (not the pixel counts) in four different images (from a single μPAD channel) was totaled together. The total average from the three different assays was 6±1. The volume of loaded sample of 5 μL, corresponding to 1 copy/μL×5 μL=5 copies, is comparable to the above count of particle clusters. It should be noted that a portion of such clusters may not represent “true” aggregation caused by antibody-antigen binding but rather non-specific aggregation. The result shown in FIG. 4 further corroborate this fact, as the pixel counts with zero concentration is ˜80, representing a small extent of non-specific aggregation, while those with 1 copy/μL is ˜280. In addition, the genome copy number (evaluated by RT-qPCR) does not truly represent the number of “all” virus particles, which can be higher. It is also possible that the sample contained free antigens and fragments in addition to intact viruses, which could also enable particle immunoagglutination.

Smartphone Microscope Assays with Field Water Samples. We then proceeded to further evaluate this method for two different field water samples: intact noroviruses were spiked into tap water and reclaimed wastewater. Water samples were serially diluted using the same tap water or reclaimed wastewater, thus the sample matrices were undiluted. As described in the Methods section, the raw images were processed to remove background noise using the cut-off intensities of the overall mean+40, +45 or +50. These images were then binarized, and further processed to remove non-aggregated particles (isolating only the aggregated particles) using the cut-off pixel areas of 30. The cut-off intensities (mean+40, +45, or +50) were selected that minimized the presence of background noise, represented by single pixels not clustered together. Particles were always represented by clusters of pixels. Experiments were repeated 6 times with both tap water and reclaimed wastewater, each time using a different μPAD.

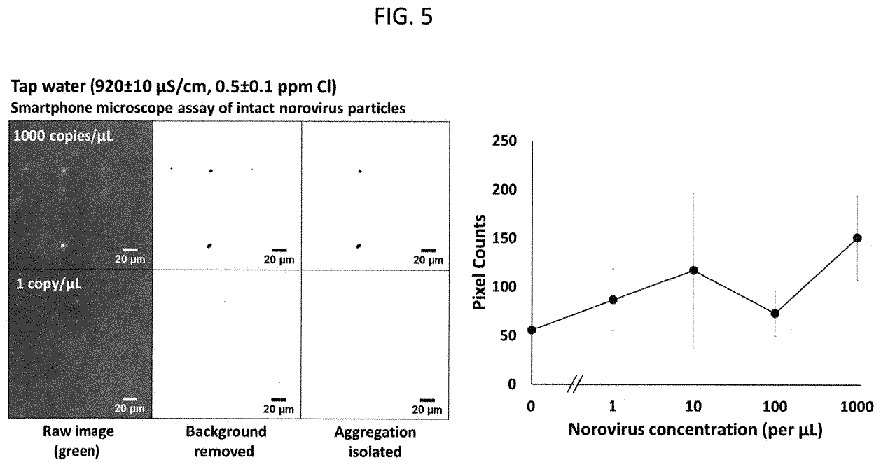

The assay results with tap water are depicted in FIG. 5. No data points passed the Wilcoxon rank sum test (p>0.05), while the p value was the smallest (0.063) with the highest concentration of 1000 copies/μL. While the overall pixel counts generally increased from the negative control, they were not significantly different. Additionally, the pixel counts are also lower (80-160) than those with DI water (270-390). These results can be attributed to electrolytes common in tap water (its conductivity was 920±10 μS/cm) or its high chlorine content (0.5±0.1 ppm).

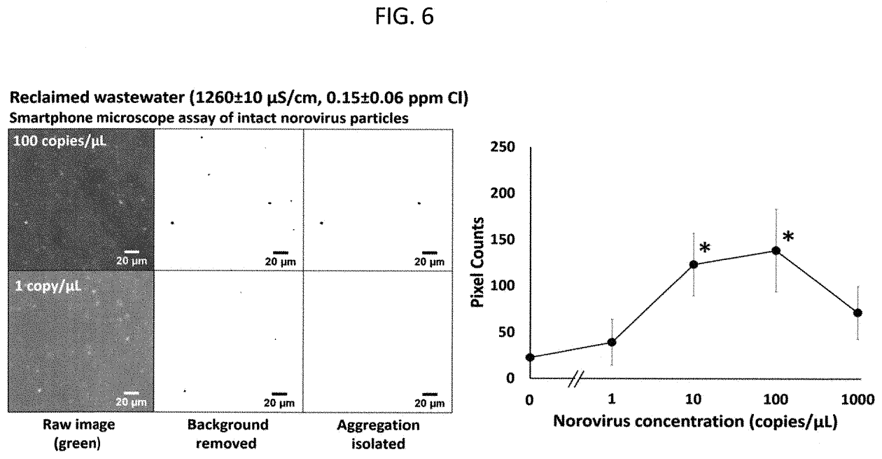

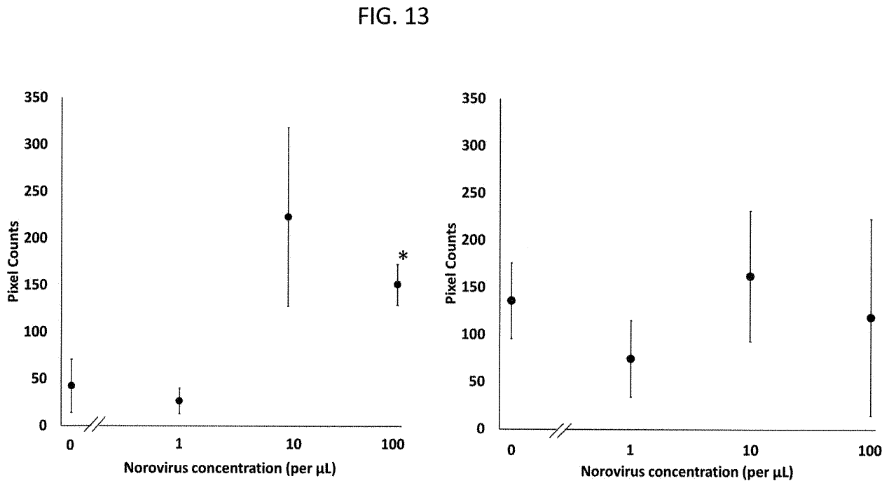

Identical experiments were repeated with reclaimed wastewater. The assay results with reclaimed wastewater are shown in FIG. 6. While the pixel counts (40-140) are still lower than those with DI water (270-390) and comparable to those of tap water (40-140), the lowest concentration that was significantly different (with Wilcoxon rank sum test) from the negative control (unspiked reclaimed wastewater) is 10 copies/μL (corresponding to 100 ag/μL), again close to the single virus copy level. The overall curve also resembles the one with DI water, i.e., an increase up to 100 copies/μL followed by a decrease at 1000 copies/μL. The conductivity of reclaimed wastewater was 1260±10 μS/cm, which was even higher than that 920±10 μS/cm of tap water, while its chlorine content was 0.15±0.06 ppm, significantly lower than that 0.5±0.1 ppm of tap water. To confirm the effect of chlorine to our assay, a control experiment was performed by adding 0.5 ppm and 5 ppm chlorine to DI water, and the results are shown in FIG. 13. Compared to the DI water results (FIG. 4), the error bars were larger and comparable to those with the tap water results (FIG. 5). With 0.5 ppm chlorine, a very narrow linear response up to 10 copies/μL was observed followed by premature saturation. Such narrow linearity could not be found with 5 ppm chlorine, one order of magnitude higher concentration than that of tap water. Thus, chlorine could be responsible for rendering the assay results less reproducible, although the role of electrolytes in tap water could not be ruled out entirely. In addition, chlorine might have adversely affected the availability of antibody-conjugated particles. (Chlorines can easily be removed by simply letting them to evaporate from water samples.) The excellent LODs in DI water and reclaimed wastewater can be attributed to many factors. Most importantly, we developed an image processing algorithm that isolated only the immunoagglutinated particles and counted the total number of such pixels. While a large number of fluorescent dyes and/or nanoparticles were necessary to collect sufficiently strong signals in other optical detection methods, only a small number of particles were necessary for individual counting. It also contributed to minimizing non-specific aggregation and facilitating capillary action-driven washing. In addition, most immunoagglutinated particles were retained and quantified in the field of view through direct imaging and counting on a paper substrate, enabling single virus copy level detection.

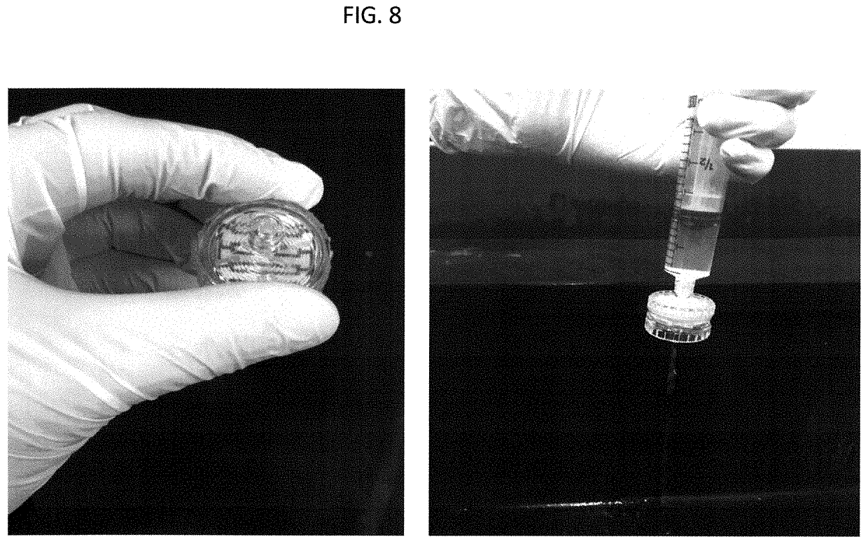

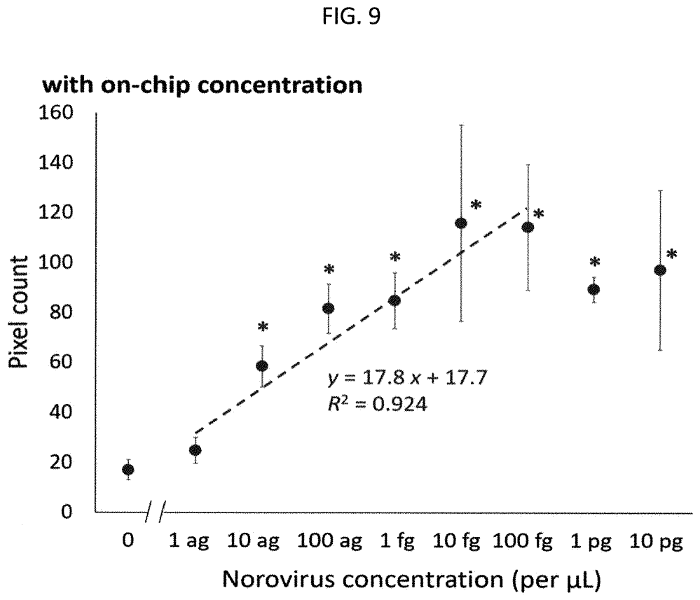

In addition, our assay can also “concentrate” norovirus directly on gAD using a syringe filter by passing 10 mL of virus target solution, to improve the sensitivity and LOD (FIG. 8). Unlike bacteria, noroviruses are too small (23-40 nm in diameter) to be captured on typical paper fibers (typical pore size is ca. 10 μm). However, negatively charged paper fibers, e.g. nitrocellulose, can capture many different virus particles through electrostatic attraction. The effect of on-chip concentration for assessing norovirus capsids using a benchtop fluorescent microscope is shown in FIG. 9. Without concentration, the LOD was 100 ag/μL as shown in FIG. 2, while the on-chip concentration decreased the LOD to 10 ag/μL. For both cases, the curves were linear up to 100 fg/μL with good linearity (R²=0.879 and 0.924, respectively) and all concentrations from 10 ag/μL to 100 fg/μL were also significantly different from zero concentration), indicating the particle aggregation was highly correlated to the norovirus presence and minimum non-specific aggregation. Further optimization of this concentration procedure, including the filtration of large volumes of water (>10 L) could definitely improve the LOD. Virus recovery efficiency with electronegative filters is not very high, requiring filtration of large volumes of water.

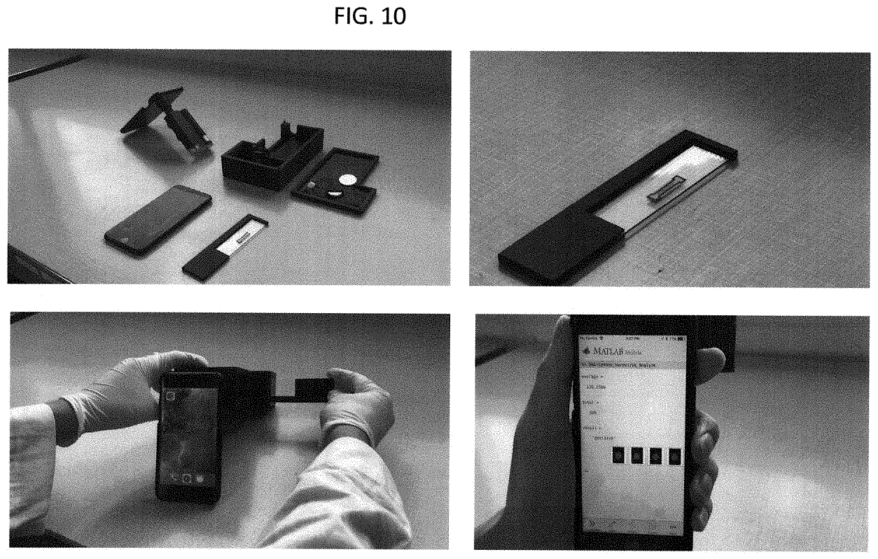

The smartphone based fluorescence microscope is further improved using the 3D-printed enclosure, where a microscope attachment to a smartphone, a blue LED light source, a button battery, and a chip holder are incorporated, as shown in FIG. 10. A suspension of antibody-conjugated fluorescence particles and a water sample that may contain norovirus are sequentially added as described in [00030], and the particle suspension and the water sample spread spontaneously to fill the length of the channel, and subsequently mixed together. One-channel chip is shown in FIG. 10, while 4-channel chips were used throughout this study. The chip is then inserted into the enclosure, and a MATLAB Mobile original code analyzes the images and count only the aggregated particles and subsequently NoV number, in the method previously described in [00032], [00034] and [00035].

It is to be understood that both the foregoing general description of the invention and the following detailed description are exemplary, and thus do not restrict the scope of the invention.

All publications mentioned herein are incorporated by reference to the extent they support the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

We claim:

1. A device for detecting and/or quantifying an enteric virus comprising a microfluidic paper analytic device (μPAD).

2. The device of claim 1, wherein said virus is a human enteric virus.

3. The device of claim 1, wherein said virus is norovirus or coronavirus.

4. The device of claim 1, wherein said device further comprises a smartphone-based fluorescence microscope comprising a smartphone, a microscope attachment, a light source (such as LED), a battery to power said light source (such as LED) (e.g., a button battery), and an optical filter.

5. The device of claim 4, where a microscope attachment, an LED, a battery to power LED, and an optical filter are housed within a plastic enclosure to block ambient lighting.

6. A method for detecting an enteric virus comprising
(a) applying a suspension comprising said virus to a microfluidic paper analytic device;
(b) adding an anti-virus conjugated fluorescent particle suspension to the microfluidic paper analytic device;
(c) allowing particles and viruses spread spontaneously throughout the μPAD channel via capillary action, allowing the particles to aggregate and facilitating imaging of individual particles.

7. The method of claim 9, wherein the virus is present in a concentration ranging from 10⁰ to 10⁵ virions.

8. The method of claim 9, wherein said virus is capable of causing disease upon ingestion of low doses ranging from 10⁰ to 10² virions.

9. The method of claim 9, wherein said measurement is taken without using any sample concentration or nucleic acid amplification step.

10. The method of claim 9, wherein said μPAD comprises nitrocellulose paper, cellulose paper, or polymeric fiber filter.

11. The method of claim 9, wherein said suspension has not been pre-purified, pre-concentrated, or pre-amplified prior to testing.

12. The method of claim 9, wherein said method involves a single virus copy level detection of said virus.

13. The method of claim 9, wherein said virus is a human enteric virus.

14. The method of claim 9, wherein said virus is norovirus or coronavirus.

15. The method of claim 9, wherein said imaging and counting aggregation of antibody-conjugated, fluorescent submicron particles is on-paper.

16. The device of claim 1, wherein said method involves a single virus copy level detection of said virus.

17. The method of claim 1, wherein said sample comprises water.

18. A kit for detecting an enteric virus comprising
a microfluidic paper analytic device (μPAD),
a suspension of antibody conjugated fluorescent particles
optionally a syringe filter to concentrate a water sample, and
a smartphone-based fluorescence microscope.

19. The kit of claim 18, wherein said virus is norovirus or coronavirus.
